(19) United States
(12) Patent Application Publication
(10) Pub. No.: US 2004/0038874 A1
(43) Pub. Date: Feb. 26, 2004

(54) Method of treatment of persistent pain

(76) Inventor: Osemwota Omoigui, Tarzana, CA (US)

Correspondence Address: Osemwota Omoigui MD, 4019 W. Rosecrans Ave., Hawthorne, CA, 90250, US

(21) Appl. No.: 10/224,743

(22) Filed: Aug. 22, 2002

(51) Int. Cl. A61K 38/00; A61K 39/395

(52) U.S. Cl. 514/12; 424/145.1

(57) ABSTRACT

This invention relates to a method for treating persistent pain disorders by inhibiting the biochemical mediators of inflammation in a subject comprising administering to said subject a therapeutically effective dosage of said inhibitor. Said process for treating persistent pain disorders is based on Sota Omoigui's Law, which states: The origin of all pain is inflammation and the inflammatory response. Biochemical mediators of inflammation that are targeted for inhibition include but are not limited to: prostaglandin, nitric oxide, tumor necrosis factor alpha, interleukin 1-alpha, interleukin 1-beta, interleukin-4, Interleukin-6 and interleukin-8, histamine and serotonin, substance P, Matrix Metallo-Proteinase, calcitonin gene-related peptide, vasoactive intestinal peptide as well as the potent inflammatory mediator peptide proteins neurokinin A, bradykinin, kallidin and T-kinin.

### BACKGROUND OF THE INVENTION

This invention relates to a method of treatment of persistent pain by application of Sota Omoigui's Law, which states: The origin of all pain is inflammation and the inflammatory response. Irrespective of the type of pain whether it is acute pain as in a sprain, sports injury or eurochange jellyfish sting or whether it is chronic pain as in arthritis, migraine pain, back or neck pain from herniated disks, RSD/CRPS pain, migraine, Fibromyalgia, Interstitial cystitis, Neuropathic pain, Post-stroke pain etc, the underlying basis is inflammation and the inflammatory response. Irrespective of the characteristic of the pain, whether it is sharp, dull, aching, burning, stabbing, numbing or tingling, all pain arise from inflammation and the inflammatory response.

### DESCRIPTION OF THE PRIOR ART

The current theories and treatment options for persistent pain are not satisfactory. The population of patients with chronic pain and disrupted lives grows constantly. According to the American Pain foundation, there are 75 million Americans who have chronic pain. Pain is the second most common reason for doctor visits. Unless we can understand how pain is generated, we cannot provide a solution. Our understanding of Pain has not advanced since the 1965 publication of the Gate Theory of Pain by Canadian psychologist Ronald Melzack and British physiologist Patrick Wall. In their paper titled “Pain Mechanisms: A New Theory”¹, Melzack and Wall suggested a gating mechanism within the spinal cord that closed in response to normal stimulation of the fast conducting “touch” nerve fibers; but opened when the slow conducting “pain” fibers transmitted a high volume and intensity of sensory signals. The gate could be closed again if these signals were countered by renewed stimulation of the large fibers. Sota Omoigui's Law is a dramatic and revolutionary shift from a focus on structural pathology to an understanding of the biochemical origin of Pain. Current medical theories place an over reliance on structural abnormalities to explain pain syndromes. This is not surprising because our current imaging technologies are structure based. Physicians are comfortable treating what they see. Patients who have structural abnormalities such as a herniated disk on MRI scans get operated upon often times needlessly and end up with more back or neck pain. Patients with severe pain who do not have structural abnormalities on MRI scans are dismissed as psychiatric cases. The fallacy of this approach has been confirmed in numerous published studies. In one of these studies², the authors performed magnetic resonance imaging on sixty-seven individuals who had never had low-back pain, sciatica, or neurogenic claudication. The scans were interpreted independently by three neuro-radiologists who had no knowledge about the presence or absence of clinical symptoms in the subjects. About one-third of the subjects were found to have a substantial abnormality. Of those who were less than sixty years old, 20 per cent had a herniated nucleus pulposus and one had spinal stenosis. In the group that was sixty years old or older, the findings were abnormal on about 57 per cent of the scans: 36 per cent of the subjects had a herniated nucleus pulposus and 21 per cent had spinal stenosis. There was degeneration or bulging of a disc at least one lumbar level in 35 per cent of the subjects between twenty and thirty-nine years old and in all but one of the sixty to eighty-year-old subjects. In view of these findings in asymptomatic subjects, the authors concluded that abnormalities on magnetic resonance images must be strictly correlated with age and any clinical signs and symptoms before operative treatment is contemplated. In another study, the authors examined the prevalence of abnormal findings on magnetic resonance imaging (MRI) scans of the lumbar spine in people without back pain. 52 percent of the asymptomatic subjects were found to have a bulge at least at one level, 27 percent had a protrusion, and 1 percent had an extrusion. Thirty-eight percent had an abnormality of more than one intervertebral disk. The prevalence of bulges, but not of protrusions, increased with age. The most common nonintervertebral disk abnormalities were Schmorl's nodes (herniation of the disk into the vertebral-body end plate), found in 19 percent of the subjects; annular defects (disruption of the outer fibrous ring of the disk), in 14 percent; and facet arthropathy (degenerative disease of the posterior articular processes of the vertebrae), in 8 percent. The findings were similar in men and women. The authors concluded that on MRI examination of the lumbar spine, many people without back pain have disk bulges or protrusions but not extrusions. The authors went further to state that given the high prevalence of these findings and of back pain, the discovery by MRI of bulges or protrusions in people with low back pain may frequently be coincidental. In another study⁴, which tracked the natural history of individuals with asymptomatic disc abnormalities in magnetic resonance imaging the authors stated that the high rate of lumbar disc alterations recently detected in asymptomatic individuals by magnetic resonance imaging demands reconsideration of a pathomorphology-based explanation of low back pain and sciatica.

The origins of pain are the biochemical mediators of inflammation. To treat pain, we must block these mediators and block the signals they send up through the nerve cells.

### SUMMARY OF THE INVENTION

The present invention provides a method for the treatment of persistent pain in a human by the use of drugs or medication that antagonize any of the biochemical mediators of inflammation. Sota Omoigui's Law of Pain states that: The origin of all pain is inflammation and the inflammatory response. . Irrespective of the type of pain whether it is acute pain as in a sprain, sports injury or eurochange jellyfish sting or whether it is chronic pain as in arthritis, migraine, back or neck pain from herniated disks, RSD/CRPS pain, Fibromyalgia, Interstitial cystitis, Neuropathic pain, Post-stroke pain etc, the underlying basis is inflammation and the inflammatory response. Irrespective of the characteristic of the pain, whether it is sharp, dull, aching, burning, stabbing, numbing or tingling, all pain arise from inflammation and the inflammatory response. On the basis of Sota Omoigui's Law of Pain, antagonism of inflammation and the inflammatory response will relieve pain of every origin, type and character.

The biochemical mediators produced by the immune cells include prostaglandin, nitric oxide, tumor necrosis factor alpha, interleukin 1-alpha, interleukin 1-beta, interleukin-4, Interleukin-6 and interleukin-8, histamine, serotonin. The biochemical mediators produced by the nerve cells include inflammatory protein Substance P, calcitonin gene-related peptide (CGRP) neurokinin A and vasoactive intestinal peptide.

Cell enzymes that catalyze reaction pathways and generate these biochemical mediators of inflammation include cyclooxygenase (COX), lipoxygenase (LOX). A cell enzyme that is activated by inflammatory mediators such as TNF-alpha and interleukin-1 is Gelatinase B or Matrix Metallo-Proteinase 9 (MMP-9). Once activated MMP-9 helps immune cells migrate through the blood vessels to inflammatory sites or to metastatic sites. Activated, MMP-9 can also degrade collagen in the extra cellular matrix of articular bone and cartilage and is associated with joint inflammation and bony erosions⁵.

Drugs and medications which inhibit these biochemical mediators of inflammation include:

Non-steroidal anti-inflammatories, such as aspirin, tolmetin sodium, indomethacin and ibuprofen, inhibit the enzyme cyclooxygenase and therefore decrease prostaglandin synthesis. Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase and lipoxygenase. The cyclooxygenase enzyme pathway results in the formation of inflammatory mediator prostaglandins and thromboxane.

Glucocorticoids are naturally occurring hormones that prevent or suppress inflammation and immune responses when administered at pharmacological doses. The anti-inflammatory corticosteroids inhibit the activation of phospholipase A₂ by causing the synthesis of an inhibitory protein called lipocortin. It is lipocortin that inhibits the activity of phospholipases and therefore limits the production of potent mediators of inflammation such as prostaglandins and leukotriene.

Botulinum toxins are potent neurotoxins which block the release of neurotransmitters. One of these transmitters called acetylcholine is released by nerve cells and transported into muscle cells to signal the muscle to contract. Blockade of this transmitter by Botulinum toxin can produce a long lasting relief of muscle spasms. Botulinum toxins also inhibit the release of tumor necrosis factor alpha⁶ (TNF-alpha) from immune cells and thus can alleviate pain and spasm produced by the inflammatory response.

Tumor Necrosis Factor Alpha Blocker Medications

The central role in inflammatory responses have Interleukin-1 and TNF-alpha, because the administration of their antagonists, such as IL-1ra (Interleukin-1 receptor antagonist), soluble fragment of Interleukin-1 receptor, or monoclonal antibodies to TNF-alpha and soluble TNF receptor, all block various acute and chronic responses in animal models of inflammatory diseases.

Etanercept (ENBREL) is a fusion protein produced by recombinant DNA technology. Etanercept binds to and inactivates Tumor Necrosis Factor (TNF-alpha) but does not affect TNF-alpha production or serum levels. Etanercept may also modulate other biologic responses that are induced or regulated by TNF-alpha such as production of adhesion molecules, other inflammatory cytokines and matrix metalloproteinase-3 (MMP-3 or stromelysin).

Infliximab is a monoclonal antibody targeted against tumor necrosis factor-alpha (TNF-alpha). Infliximab neutralizes the biological activity of the cytokine tumor necrosis factor-alpha (TNF-alpha). Infliximab binds to high affinity soluble and transmembrane forms of TNF-alpha and inhibits the binding of TNF-alpha with its receptors. Infliximab does not neutralize TNF-beta, a related cytokine that utilizes the same receptors as TNF-alpha. Biological activities attributed to TNF-alpha include induction of pro-inflammatory cytokines such as interleukin (IL)-1 and IL-6; enhancement of leukocyte migration by increasing endothelial layer permeability; expression of adhesion molecules by endothelial cells and leukocytes; activation of neutrophil and eosinophil functional activity; fibroblast proliferation; synthesis of prostaglandins; and induction of acute phase and other liver proteins.

Anakinra is a form of the human interleukin-1 receptor antagonist (IL-1Ra) produced by recombinant DNA technology. Anakinra differs from the naturally occurring native human IL-1Ra in that it has an additional methionine residue at its amino terminus. Anakinra acts similarly to the naturally occurring interleukin-1 receptor antagonist (IL-1Ra). IL-1Ra blocks effects of Interleukin-1 by competitively inhibiting binding of this cytokine, specifically IL-alpha and IL-beta, to the interleukin-1type 1 receptor (IL-1R1), which is produced in a wide variety of tissues. IL-1Ra is part of the feedback loop that is designed to balance the effects of inflammatory cytokines.

Leflunomide interferes with RNA and protein synthesis in immune T and B-lymphocytes. T and B cell collaborative actions are interrupted and antibody production is suppressed. Leflunomide is the first agent for rheumatoid arthritis that is indicated for both symptomatic improvement and retardation of structural joint damage. Leflunomide may also have anti-inflammatory properties secondary to reduction of histamine release, and inhibition of induction of cyclooxygenase-2 enzyme (COX-2). Leflunomide may decrease proliferation, aggregation and adhesion of peripheral and joint fluid mononuclear cells. Decrease in the activity of immune lymphocytes leads to reduced cytokine and antibody-mediated destruction of joints and attenuation of the inflammatory process.

Phosphodiesterase inhibitors such as Pentoxifylline have other unique effects. The drugs suppress inflammatory cytokine production by T cells and macrophages⁷. Some of the anti-inflammatory effects occurs by blocking nitric oxide (NO) production by macrophages. Pentoxifylline also blocks the production of Tumor Necrosis Factor Alpha. In one study, Pentoxifylline prevented nerve root injury and swelling (dorsal root ganglion compartment syndrome) caused by topical application of disk tissue (nucleus pulposus)⁸

Tetracyclines such as doxycycline and minocycline may block a number of cytokines including Interleukin-1⁹¹⁰, IFNg¹¹, NO-synthetases, and metalloproteinases¹². Interleukin-1 and IFN-.gamma act synergistically with TNF-alpha and are known to be toxic to nerve tissue^{13 14151617}.

5-HT3-receptor antagonist medications such as Ondansetron diminish serotonin-induced release of substance P from C-fibers and prevent unmasking of NK2-receptors in the presence of serotonin¹⁸.

Bisphosphonates medications such as Pamidronate reduce bone complications and related pain in patients with Paget's disease, osteoporosis and bone metastasis, thereby improving quality of life. Bisphosphonates have intrinsic anti-tumor activity by virtue of inducing tumor cell adherence to marrow, reducing interleukin-6 secretion, inducing tumor cell apoptosis, or inhibiting angiogenesis¹⁹

Anti-depressant medication such as Amitriptyline also have effects on inflammatory mediators. Prolonged administration of amitriptyline and desipramine have resulted in a significant increase in the secretion of the anti-inflammatory cytokine Interleukin-10²⁰.

Anti-seizure medications such as Oxcarbazepine or Zonisamide decrease pain by reducing the rate of continuing discharge of injured and inflamed nerve fibers. Blockade of sodium channels in nerve cells leads to a decrease in electrical activity and a subsequent reduction in release of the excitatory nerve transmitter glutamate. Anti-seizure drugs also inhibit the initiation and propagation of painful nerve impulses by inhibiting Nitric Oxide Synthetase activity²¹. Nitric Oxide Synthetase is the enzyme responsible for the production of the inflammatory mediator Nitric Oxide. Anti-seizure drugs may also protect nerve cells from free radical damage by Nitric Oxide and/or hydroxyl radicals (OH*)²²

Thalidomide and analogues mainly inhibit tumor necrosis factor alpha (TNF-alpha) synthesis but the drugs also have effects on other cytokines. Thalidomides increase the production of the anti-inflammatory cytokine interleukin-10 (IL-10) in lesioned sciatic nerves. In addition, Thalidomides stimulate the release of the pain relieving natural opioid peptide methionine-enkephalin in the dorsal horn of the spinal cord²³

### DETAILED DESCRIPTION

The origin of pain are the biochemical mediators of inflammation and the inflammatory response. To treat pain, we must block these mediators and block the signals they send up through the nerve cells. We can now measure many of these inflammatory mediators in the blood and spinal fluid. However, our current technology does not allow us to image these mediators. Hopefully sometime in the future we will be able to do so.

Inflammation occurs when there is infection or tissue injury. Tissue injury may arise from a physical, chemical or biological trauma or irritation. Degeneration of tissue subsequent to aging or previous injury can also lead to inflammation. Injured tissues can be muscle, ligament, disks, joints or nerves. A variety of mediators are generated by tissue injury and inflammation. These include substances produced by damaged tissue, substances of vascular origin as well as substances released by nerve fibers themselves, sympathetic fibers and various immune cells²⁴. There are three phases of an inflammatory response: initiation, maintenance and termination. Upon tissue injury or painful stimulation, specialized blood cells in the area such as basophils, mast cells and platelets release inflammatory mediators serotonin, histamine and nitric oxide. Subsequent to the binding of serotonin to its receptor, there is inflammation of the adjacent nerves and the nerve endings release short-lived inflammatory peptide proteins such as substance P, Calcitonin gene-related peptide (CGRP). In addition, clotting factors in the blood produce and activate potent inflammatory mediator peptide proteins called neurokinin A, bradykinin, kallidin and T-kinin. All of these proteins increase blood flow to the area of injury, stimulate arachidonic acid metabolism to generate inflammatory mediators prostaglandins and attract specialized immune cells to the area. The first immune cells to the area are tissue macrophages, which provide the front line defense against bacterial infection. Macrophages release powerful enzymes to digest any bacteria that are present and produce potent inflammatory chemical mediators (called cytokines) to attract and activate other cells of the immune system. Shortly thereafter the area of bacterial invasion or tissue injury is invaded by the other immune cells, which include white blood cells such as T helper cells, lymphocytes, neutrophils, eosinophils, and other cells such as fibroblasts and endothelial cells. These immune cells respond to the chemical mediators, release destructive enzymes to kill any invading organism and release more chemical mediators to attract more immune cells. A consequence of this immune response is tissue damage, pain and spasm. In a sense the initial immune reaction ignites a cascade of immune reactions and generates an inflammatory soup of chemical mediators. These chemical mediators produced by the immune cells include prostaglandin, nitric oxide, tumor necrosis factor alpha, interleukin 1- alpha, interleukin 1-beta, interleukin-4, Interleukin-6 and interleukin-8, histamine, serotonin, In the area of injury and subsequently in the spinal cord, enzymes such as cyclooxygenase increase the production of these inflammatory mediators. These chemical mediators attract tissue macrophages and white blood cells to localize in an area to engulf (phagocytize) and destroy foreign substances. The chemical mediators released during the inflammatory response give rise to the typical findings associated with inflammation.

Effects of the Inflammatory Response.

The primary physical effect of the inflammatory response is for blood circulation to increase around the affected area. Blood vessels around the site of inflammation dilate, allowing increased blood flow to the area. Gaps appear in the cell walls surrounding the area, allowing the larger cells of the blood, i.e. the immune cells, to pass through. As a result of the increased blood flow, the immune presence is increased. All of the different types of cells that constitute the immune system congregate at the site of inflammation, along with a large supply of chemical mediators, which fuel the immune response. There is an increase in local or body heat. The main symptoms of the inflammatory response are as follows.

1. The tissues in the area are red and warm, as a result of the large amount of blood reaching the site.

2. The tissues in the area are swollen, again due to the increased amount of blood and proteins that are present.

3. The tissues in the area are painful, due to the presence of the inflammatory mediators and due to the expansion of tissues, causing mechanical pressure on nerve cells.

Effects of the Inflammatory Mediators

The inflammatory mediators activate local pain receptors and nerve terminals and produce hypersensitivity in the area of injury. Activity of the mediators results in excitation of pain receptors in the skin, ligaments, muscle, nerves and joints. Excitation of these pain receptors stimulate the specialized nerves e.g. C fibers and A-delta fibers that carry pain impulses to the spinal cord and brain. Subsequent to tissue injury, the expression of sodium channels in nerve fibers is altered significantly thus leading to abnormal excitability in the sensory neurons. Nerve impulses arriving in the spinal cord stimulate the release of inflammatory protein Substance P. The presence of Substance P and other inflammatory proteins such as calcitonin gene-related peptide (CGRP) neurokinin A and vasoactive intestinal peptide removes magnesium induced inhibition and enables excitatory Inflammatory proteins such as glutamate and aspartate to activate specialized spinal cord NMDA receptors. This results in magnification of all nerve traffic and pain stimuli that arrive in the spinal cord from the periphery. Activation of motor nerves that travel from the spinal cord to the muscles results in excessive muscle tension. More inflammatory mediators are released which then excite additional pain receptors in muscles, tendons and joints generating more nerve traffic and increased muscle spasm. Persistent abnormal spinal reflex transmission due to local injury or even inappropriate postural habits may then result in a vicious circle between muscle hypertension and pain²⁵. Separately, constant C-fiber nerve stimulation to transmission pathways in the spinal cord resulting in even more release of inflammatory mediators but this time within the spinal cord. Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E₂ (PGE₂) levels in the cerebrospinal fluid. The major inducer of central Cox-2 upregulation is inflammatory mediator interleukin-1^{β} in the CNS²⁶. Basal levels of the enzyme phospholipase A₂ activity in the CNS do not change with peripheral inflammation. Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal ‘baskets’ may underlie painful sympathetic-sensory coupling²⁷. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5). The central nervous system response to pain can keep increasing even though the painful stimulus from the injured tissue remains steady. This “wind-up” phenomenon in deep dorsal neurons can dramatically increase the injured person's sensitivity to the pain. Local tissue inflammation can also result in pain hypersensitivity in neighboring uninjured tissue (secondary hyperalgesia) by spread and diffusion of the excess inflammatory mediators that have been produced as well as by an increase in nerve excitability in the spinal cord (central sensitization). This can result in a syndrome comprising diffuse muscle pain and spasm, joint pain, fever, lethargy and anorexia.

The Complex Interaction of Inflammatory Mediators

The inflammatory mediators interact in a complex way to induce, enhance and propagate persistent pain. There are also natural anti-inflammatory mediators produced by the body to cool down inflammation and the inflammatory response.

Interleukin-1 beta is a potent pain-generating mediator. Two pain producing pathways have been identified: Inflammatory stimuli or injury to soft tissue induces the production of mediator Bradykinin, which stimulates the release of mediator Tumor necrosis factor alpha. The TNF-alpha induces production of (i) Interleukin-6 and Interleukin-1-Beta which stimulate the production of cyclooxygenase enzyme products, and (ii) Inflammatory mediator Interleukin-8, which stimulates production of sympathomimetics (sympathetic hyperalgesia)²⁸. Effects of Interleukin-1 beta include:

Interleukin-1 beta stimulates inflammatory mediators prostaglandin E₂ (PGE₂), cyclooxygenase-2 (COX-2) and matrix metalloproteases (MMPs) production^{29, 30}

Interleukin-1 beta is a significant catalyst in cartilage damage. It induces the loss of proteoglycans, prevents the formation of the cartilage matrix³¹ and prevents the proper maintenance of cartilage.

Interleukin-1 beta is a significant catalyst in bone resorption It stimulates osteoclasts cells involved in the resorption and removal of bone³²³³³⁴

Interleukin-6

This is another potent pain-generating inflammatory mediator. A significant amount of InterLeukin-6 is produced in the rat spinal cord following peripheral nerve injury that results in pain behaviors suggestive of neuropathic pain. These spinal IL-6 levels correlated directly with the mechanical allodynia intensity following nerve injury³⁵.

Interleukin-8

This is a pain-generating inflammatory mediator. In one study of patients with post herpetic neuralgia, the patients who received methylprednisolone, had interleukin-8 concentrations decrease by 50 percent, and this decrease correlated with the duration of neuralgia and with the extent of global pain relief³⁶ (p<0.001 for both comparisons).

Interleukin-10

This is one of the natural anti-inflammatory cytokines, which also include Interleuken-1 receptor antagonist (IL-1ra), Interleukin-4, Interleukin-13 and transforming growth factor-betal (TGF-betal). Interleukin-10 (IL-10) is made by immune cells called macrophages during the shut-off stage of the immune response. Interleukin-10 is a potent anti-inflammatory agent, which acts partly by decreasing the production of inflammatory cytokines interleukin-1 beta (Interleukin-1 beta), tumor necrosis factor-alpha (TNF-alpha) and inducible nitric oxide synthetase (iNOS), by injured nerves and activated white blood cells, thus decreasing the amount of spinal cord and peripheral nerve damage³⁷³⁸. In rats with spinal cord injury (SCI), a single injection of IL-10 within half an hour resulted in 49% less spinal cord tissue loss than in untreated rats. The researchers observed nerve fibers traveling straight through the spared tissue regions, across the zone of injury. They also reported a decrease in the inflammatory mediator TNF-alpha, which rises significantly after SCI.

Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase (COX) and lipoxygenase (LOX). These two enzyme pathways compete with one another. The cyclooxygenase enzyme pathway results in the formation of inflammatory mediator prostaglandins and thromboxane. The lipoxygenase enzyme pathway results in the formation of inflammatory mediator leukotriene. Because they are lipid soluble these mediators can easily pass out through cell membranes.

In the cyclooxygenase pathway, the prostaglandins D, E and F plus thromboxane and prostacyclin are made. Thromboxanes are made in platelets and cause constriction of vascular smooth muscle and platelet aggregation. Prostacyclins, produced by blood vessel walls, are antagonistic to thromboxanes as they inhibit platelet aggregation.

Prostaglandins have diverse actions dependent on cell type but are known to generally cause smooth muscle contraction. They are very potent but are inactivated rapidly in the systemic circulation. Leukotrienes are made in leukocytes and macrophages via the lipoxygenase pathway. They are potent constrictors of the bronchial airways. They are also important in inflammation and hypersensitivity reactions as they increase vascular permeability and attract leukocytes.

Tumor necrosis factor alpha—This inflammatory mediator is released by macrophages as well as nerve cells. Very importantly, TNF-alpha is released from injured or herniated disks. During an inflammatory response, nerve cells communicate with each other by releasing neuro-transmitter glutamate. This process follows activation of a nerve cell receptor called CXCR4 by the inflammatory mediator stromal cell-derived factor 1 (SDF-1). An extraordinary feature of the nerve cell communication is the rapid release of inflammatory mediator tumor necrosis factor-alpha (TNF alpha). Subsequent to release of TNF-alpha, there is an increase in the formation of inflammatory mediator prostaglandin. Excessive prostaglandin release results in an increased production of neurotransmitter glutamate and an increase in nerve cell communication resulting in a vicious cycle of inflammation There is excitation of pain receptors and stimulation of the specialized nerves e.g. C fibers and A-delta fibers that carry pain impulses to the spinal cord and brain.

Studies have established that herniated disk tissue (nucleus pulposus) produces a profound inflammatory reaction with release of inflammatory chemical mediators. Disk tissue applied to nerves may induce a characteristic nerve sheath injury³⁹⁴⁰⁴¹ increased blood vessel permeability, and blood coagulation. The primary inflammatory mediator implicated in this nerve injury is Tumor necrosis factor-alpha but other mediators including Interleukin 1-beta may also participate in the inflammatory reaction. Recent studies have also shown that that local application of nucleus pulposus may induce pain-related behavior in rats, particularly hypersensitivity to heat and other features of a neuropathic pain syndrome.

Nitric Oxide—This inflammatory mediator is released by macrophages. Other mediators of inflammation such as reactive oxygen products and cytokines, considerably contribute to inflammation and inflammatory pain by causing an increased local production of Cyclooxygenase enzyme. The cyclooxygenase enzyme pathway results in the formation of inflammatory mediator prostaglandins and thromboxane. Concurrently to the increased production of the Cyclooxygenase-2 (COX-2) gene, there is increased production of the gene for the enzyme inducible nitric oxide synthetase (iNOS), leading to increased levels of nitric oxide (NO) in inflamed tissues. In these tissues, NO has been shown to contribute to swelling, hyperalgesia (heightened reaction to pain) and pain. NO localized in high amounts in inflamed tissues has been shown to induce pain locally and enhances central as well as peripheral stimuli. Inflammatory NO is thought to be synthesized by the inducible isoform of nitric oxide synthetase (iNOS).

Substance P (sP)—An important early event in the induction of neuropathic pain states is the release of Substance P from injured nerves which then increases local Tumor Necrosis Factor alpha (TNF-alpha) production. Substance P and TNF-alpha then attract and activate immune monocytes and macrophages, and can activate macrophages directly. Substance P effects are selective and Substance P does not stimulate production of Interleukin-1, Interleukin-3, or Interleukin-6. Substance P and the associated increased production of TNF-alpha has been shown to be critically involved in the pathogenesis of neuropathic pain states. TNF protein and message are then further increased by activated immune macrophages recruited to the injury site several days after the primary injury. TNF-alpha can evoke spontaneous electrical activity in sensory C and A-delta nerve fibers that results in low-grade pain signal input contributing to central sensitization. Inhibition of macrophage recruitment to the nerve injury site, or pharmacologic interference with TNF-alpha production has been shown to reduce both the neuropathologic and behavioral manifestations of neuropathic pain states⁴²

Gelatinase B or Matrix Metallo-Proteinase 9 (MMP-9)—This enzyme is one of a group of metalloproteinases (which includes collagenase and stromelysin) that are involved in connective tissue breakdown. Normal cells produce MMP-9 in an inactive, or latent form. The enzyme is activated by inflammatory mediators such as TNF-alpha and interleukin-1 that are released by cells of the immune system (mainly neutrophils but also macrophages and lymphocytes) and transformed cells⁴³⁴⁴. MMP-9 helps these cells migrate through the blood vessels to inflammatory sites or to metastatic sites. Activated, MMP-9 can also degrade collagen in the extra cellular matrix of articular bone and cartilage and is associated with joint inflammation and bony erosions⁴⁵. Consequently, MMP-9 plays a major role in acute and chronic inflammation, in cardiovascular and skin pathologies as well as in cancer metastasis. MMP-9 can also degrade a protein called beta amyloid. Normal cells produce MMP-9 in an inactive, or latent form, converting it to active enzyme when it is needed. But when normal brain cells producing MMP-9 fail to activate the enzyme, insoluble amyloid-b could accumulate in brain tissue. Previous research has shown that the undegraded form of amyloid-beta accumulates in the brain as senile “plaques” that signal the presence of Alzheimer's disease⁴⁶.

### Natural Suppression of the Inflammatory Response

How does the inflammatory response end?

Immune cells produce anti-inflammatory cytokine mediators that help to suppress the inflammatory response and suppress the production of pro-inflammatory cytokines. The natural anti-inflammatory cytokines are Interleuken-1 receptor antagonist (IL-1ra), Interleukin-10, Interleukin-4, Interleukin-13 and transforming growth factor-betal (TGF-betal). Research has shown that administration of these anti-inflammatory cytokines prevents the development of painful nerve pain that is produced by a naturally occurring irritant protein called Dynorphin A⁴⁷

Under normal circumstances,, the inflammatory response should only last for as long as the infection or the tissue injury exists. Once the threat of infection has passed or the injury has healed, the area should return to normal existence.

One of the ways that the inflammatory response ends is by a phenomenon known as “Apoptosis”.

Most of the time, cells of the body die by being irreparably damaged or by being deprived of nutrients. This is known as Necrotic death. However, cells can also be killed in another way, i.e. by “committing suicide”. On receipt of a certain chemical signal, most cells of the body can destroy themselves. This is known as Apoptotic death. There are two main ways in which cells can commit Apoptosis.

1. By receiving an Apoptosis signal. When a chemical signal is received that indicates that the cell should kill itself, it does so.

2. By not receiving a “stay-alive” signal. Certain cells, once they reach an activated state, are primed to kill themselves automatically within a certain period of time, i.e. to commit Apoptosis, unless instructed otherwise. However, there may be other cells that supply them with a “stay-alive” signal, which delays the Apoptosis of the cell. It is only when the primed cell stops receiving this “stay-alive” signal that it kills itself.

The immune system employs method two above. The immune cells involved in the inflammatory response, once they become activated, are primed to commit Apoptosis. Helper T cells emit the stay-alive signal, and keep emitting the signal for as long as they recognize foreign antigens or a state of injury in the body, thus prolonging the inflammatory response. It is only when the infection or injury has been eradicated, and there is no more foreign antigen that the helper T cells stop emitting the stay-alive signal, thus allowing the cells involved in the inflammatory response to die off.

If foreign antigen is not eradicated from the body or the injury has not healed, or the helper T cells do not recognize that fact, or if the immune cells receive the stay-alive signal from another source, then chronic inflammation may develop.

The final pathway for the natural suppression of the inflammatory response is in the spinal cord where there is a complex network of inhibitory neurons (‘gate control’) that is driven by descending projections from brain stem sites. These inhibitory neurons act to dampen and counteract the spinal cord hyper excitability produced by tissue or nerve injury. Thus, peripherally evoked pain impulses pass through a filtering process involving inhibitory transmitters gamma-aminobutyric acid (GABA), glycine and enkephalins. The activity of these substances in the spinal cord usually attenuates and limits the duration of pain. In the case of persistent pain, there is evidence of pathological reduction of the supraspinal inhibitory actions in combination with ectopic afferent input in damaged nerves⁴⁸.

### Inflammatory Pain Syndromes

### Arthritis

Arthritis means inflammation of the joints. People of all ages including children and young adults can develop arthritis. The symptoms are intermittent pain, swelling, redness and stiffness in the joints. There are many different types of arthritis, some of which are rheumatoid arthritis, osteoarthritis, infectious arthritis and spondylitis. In rheumatoid arthritis, and other autoimmune diseases like systemic lupus erythematosus (SLE), the joints are destroyed by the immune system. In Osteoarthritis, the biggest risk factor is a previous injury to the joint, ligament or cartilage. Injuries that seem to heal perfectly well appear to set up a process of deterioration that can produce severe pain and disability decades later. The injury need not be sustained in one episode. Long term or repeated trauma can have the same effect. TNF-alpha and Interleukin 1-beta play an important role in rheumatoid arthritis by mediating cytokines that cause inflammation and joint destruction. TNF-alpha, Interleukin 1-beta and Substance P are elevated in the joint fluids in patients with rheumatoid arthritis⁴⁹. These inflammatory mediators are also elevated in the joint fluid in patients with osteoarthritis albeit to a far less extent. Along with mechanical factors, growth factors and cytokines such as TGF beta 1, IL-1 alpha, IL-1 beta and TNF-alpha may be involved in the formation and growth of osteophytes, since these molecules can induce growth and differentiation of mesenchymal cells. The incidence and size of osteophytes may be decreased by inhibition of direct or indirect effects of these cytokines and growth factors on osteoid deposition in treated animals⁵⁰⁵¹. Inhibition of Interleukin-1 receptor also decreases the production of metalloproteinase enzymes collagenase-1and stomelysin-1 in the synovial membrane and cartilage. These enzymes are involved in connective tissue breakdown⁵².

### Back and Neck Pain

Back and neck pain most commonly results from injury to the muscle, disk, nerve, ligament or facet joints with subsequent inflammation and spasm. Degeneration of the disks or joints produces the same symptoms and occurs subsequent to aging, previous injury or excessive mechanical stresses that this region is subjected to because of its proximity to the sacrum in the lower back.

Herniated disk tissue (nucleus pulposus) produces a profound inflammatory reaction with release of inflammatory chemical mediators most especially Tumor Necrosis Factor Alpha. Subsequent to release of TNF-alpha, there is an increase in the formation of inflammatory mediator prostaglandin and Nitric Oxide. It is now known that Tumor Necrosis Factor Alpha is released by herniated disk tissue (nucleus pulposus), and is primarily responsible for the nerve injury and behavioral manifestations of experimental sciatica associated with herniated lumbar discs⁵³. This has been confirmed by numerous animal studies and research wherein application of disk tissue (nucleus pulposus) to a nerve results in nerve fiber injury, with reduction of nerve conduction velocity, intracapillary thrombus formation, and the intraneural edema formation⁵⁴⁵⁵. One study demonstrated that disk tissue (nucleus pulposus) increases inducible nitric oxide synthetase activity in spinal nerve roots and that nitric oxide synthetase inhibition reduces nucleus pulposus-induced swelling and prevents reduction of nerve-conduction velocity. According to the authors, the results suggest that nitric oxide is involved in the pathophysiological effects of disk tissue (nucleus pulposus) in disc herniation⁵⁶. Tumor Necrosis Factor Alpha and other inflammatory mediators induce phospholipase A2 activation. High levels of phospholipase A2 previously have been demonstrated in a small number of patients undergoing lumbar disc surgery. Phospholipase A2 is the enzyme responsible for the liberation of arachidonic acid from cell membranes at the site of inflammation and is considered to be the limiting agent in the production of inflammatory mediator prostaglandins and leukotrienes⁵⁷. Subsequent to the release of inflammatory mediators, activation of motor nerves that travel from the spinal cord to the muscles results in excessive muscle tension, spasm and pain. The vast majority of herniated disk pain is inflammatory in origin, can be treated medically and does not require surgery. Surgery is only indicated when there is compression of the nerve roots producing significant muscle weakness and or urinary or bowel incontinence.

### Fibromyalgia

Fibromyalgia is a chronic, painful musculoskeletal disorder characterized by widespread pain, pressure hyperalgesia, morning stiffness, sleep disturbances including restless leg syndrome, mood disturbances, and fatigue. Other syndromes commonly associated with fibromyalgia include irritable bowel syndrome, interstitial cystitis, migraine headaches, temporomandibular joint dysfunction, dysequilibrium including nerve mediated hypotension, sicca syndrome, and growth hormone deficiency. Fibromyalgia is accompanied by activation of the inflammatory response system, without immune activation⁵⁸. In fact, there is some evidence that fibromyalgia is accompanied by some signs of immunosuppression⁵⁹. Several studies have shown that there are increased levels of the inflammatory transmitter Substance P (SP) and calcitonin gene related peptide (CGRP) in the spinal fluid of patients with fibromyalgia syndrome (FMS)⁶⁰⁶¹⁶². The levels of platelet serotonin are also abnormal⁶³. Furthermore, in patients with fibromyalgia, the level of pain intensity is related to the spinal fluid level of arginine, which is a precursor to the inflammatory mediator nitric oxide (NO)⁶⁴. Another study found increases over time in blood levels of cytokines Interleukin-6, Interleukin-8 and Interleukin-1R antibody (IL-1Ra) whose release is stimulated by substance P. The study authors concluded that because Interleukin-8 promotes sympathetic pain and Interleukin-6 induces hypersensitivity to pain, fatigue and depression, both cytokines play a role in producing FM symptoms⁶⁵.

### Interstitial Cystitis

Interstitial cystitis is a severe debilitating bladder disease characterized by unrelenting pelvic pain and urinary frequency. This sterile painful bladder disorder is associated with a defective glycosaminoglycan bladder mucosal layer and an increased number of activated mast cells. Mast cells are ubiquitous cells derived from the bone marrow and are responsible for allergic reactions as they release numerous vasodilatory, nociceptive and pro-inflammatory mediators in response to immunoglobulin E (IgE) and specific antigen. Mast cell secretion is also triggered by a number of peptides, such as bradykinin and substance P, and may also be involved in the development of inflammatory responses⁶⁶. SP-containing nerve fibres are increased in the submucosa of the urinary bladder of interstitial cystitis (IC) patients and are frequently seen in juxtaposition to Mast cells⁶⁷⁶⁸. There is enhanced sympathetic innervation of the bladder in the submucosa and detrusor muscle. In interstitial cystitis the number of neurons positive for inflammatory mediator vasoactive intestinal polypeptide and neuropeptide Y is higher⁶⁹. Substance P (SP) and bradykinin (BK) influence the excitatory motor innervation of the urinary bladder. These peptides potentiate the responses to the purinergic component of the neurogenic stimulation (that part of the contractile response that remains after treatment with atropine) and potentiate the responses to exogenously applied adenosine triphosphate (ATP)⁷⁰. Significant elevations in Interleuken-2, Interleukin-6, and Interleukin-8 have also been found in the urine of subjects with active interstitial cystitis compared with subjects with interstitial cystitis in remission and control subjects⁷¹

### Migraine

Migraine headache is caused by activation of trigeminal sensory fibers by known and unknown migraine triggers. There is subsequent release of inflammatory mediators from the trigeminal nerve. This leads to distention of the large meningeal blood vessels in the skull and brain and the development of a central sensitization within the trigeminal nucleus caudalis (TNC). Genetic abnormalities may be responsible for altering the response threshold to migraine specific trigger factors in the brain of a migraineur compared to a normal individual⁷².

The painful neurogenic vasodilation of meningeal blood vessels is a key component of the inflammatory process during migraine headache. The cerebral circulation is supplied with two vasodilator systems: the parasympathetic system storing vasoactive intestinal peptide, peptide histidine isoleucine, acetylcholine and in a subpopulation of nerves neuropeptide Y, and the sensory system, mainly originating in the trigeminal ganglion, storing inflammatory mediator substance P, neurokinin A and calcitonin gene-related peptide (CGRP)⁷³. A clear association between migraine and the release of inflammatory mediator calcitonin gene-related peptide (CGRP) and substance P (SP) has been demonstrated. Jugular plasma levels of the potent vasodilator, calcitonin gene-related peptide (CGRP) have been shown to be elevated in migraine headache. CGRP-mediated neurogenic dural vasodilation is blocked by anti-migraine drug dihydroergotamine, triptans, and opioids⁷⁴. In cluster headache and in chronic paroxysmal hemicrania, there is additional release of inflammatory mediator vasoactive intestinal peptide (VIP) in association with facial symptoms (nasal congestion, runny nose)⁷⁵. Immunocytochemical studies have revealed that cerebral blood vessels are invested with nerve fibers containing inflammatory mediator neuropeptide Y (NPY), vasoactive intestinal peptide (VIP), peptide histidine isoleucine (PHI), substance P (SP), neurokinin A (NKA), and calcitonin gene-related peptide (CGRP). In addition, there are studies reporting the occurrence of putative neurotransmitters such as cholecystokinin, dynorphin B, galanin, gastrin releasing peptide, vasopressin, neurotensin, and somatostatin. The nerves occur as a longitudinally oriented network around large cerebral arteries. There is often a richer supply of nerve fibers around arteries than veins. The origin of these nerve fibers has been studied by retrograde tracing and denervation experiments. These techniques, in combination with immunocytochemistry, have revealed a rather extensive innervation pattern. Several ganglia, such as the superior cervical ganglion, the sphenopalatine ganglion, the otic ganglion, and small local ganglia at the base of the skull, contribute to the innervation. Sensory fibers seem to derive from the trigeminal ganglion, the jugular-nodose ganglionic complex, and from dorsal root ganglia at the cervical spine level C2. The noradrenergic and most of the NPY fibers derive from the superior cervical ganglion. A minor population of the NPY-containing fibers contains vasoactive intestinal peptide (VIP), instead of NA and emanates from the sphenopalatine ganglion. The cholinergic and the vasoactive intestinal peptide (VIP)-containing fibers derive from the sphenopalatine ganglion, the otic ganglion, and from small local ganglia at the base of the skull. Most of the substance P (SP-), neurokinin A (NKA), and calcitonin gene-related peptide (CGRP)-containing fibers derive from the trigeminal ganglion. Minor contributions may emanate from the jugular-nodose ganglionic complex and from the spinal dorsal root ganglia. Neuropeptide Y (NPY), is a potent vasoconstrictor in vitro and in situ. Vasoactive intestinal peptide (VIP), peptide histidine isoleucine (PHI), substance P (SP), neurokinin A (NKA), and calcitonin gene-related peptide (CGRP) act via different mechanisms to induce cerebrovascular dilatation⁷⁶. Meningeal blood vessels are involved in the generation of migraine pain and other headaches. Classical experiments have shown that blood vessels of the cranial dura mater are the most pain-sensitive intracranial structures. Dural blood vessels are supplied by trigeminal nerve fibers, and dilate in response to activation of the trigeminal nerves and release of neuropeptide cytokines such as substance P (SP) and calcitonin gene-related peptide (CGRP)⁷⁷. CGRP can be released experimentally from dural nerve fibers, and there is evidence that this occurs also during migraine attacks. Stimulation of dural nerve fibers causes vasodilatation and an increase in dural arterial flow, which depends on the release of CGRP but not SP. SP, on the other hand, is known to mediate plasma leakage (extravasation) from small veins in the dura mater. The dural arterial flow depends also on the formation of cell wall nitric oxide. The introduction of serotonin (5-HT₁) receptor agonists such as sumatriptan changed the treatment strategies for migraine. Sumatriptan and other triptans may inhibit the release of inflammatory mediators from the trigeminal nerve. Sumatriptan has been shown to block the release of vasoactive cytokines from trigeminal nerves that surround the blood vessels in the dura mater during migraine. Sumatriptan blocks nerve fiber induced plasma extravasation but has only minor effects on nerve fiber mediated vasodilatation and dural arterial flow. Foods like cheese, beer, and wine can also induce migraine in some people because they contain the mediator histamine and/or mediator-like compounds that cause blood vessels to expand. Women tend to react to histamine-containing foods more frequently than men do, on account of a deficiency in an enzyme (diamine oxidase) that breaks histamine down. Taking supplemental B6 has been shown to be helpful in migraine, as it can increase diamine oxidase activity.

### Nerve (Neuropathic) Pain Syndromes (e.g. carpal tunnel syndrome, trigeminal neuralgia, post herpetic neuralgia, phantom limb pain)

Nociceptive pain is mediated by receptors on A-delta and C nerve fibers, which are located in skin, bone, connective tissue, muscle and viscera. These receptors serve a biologically useful role at localizing noxious chemical, thermal and mechanical stimuli. Nociceptive pain can be somatic or visceral in nature. Somatic pain tends to be well-localized, constant pain that is described as sharp, aching, throbbing, or gnawing. Visceral pain, on the other hand, tends to be vague in distribution, spasmodic in nature and is usually described as deep, aching, squeezing and colicky in nature. Examples of nociceptive pain include: post-operative pain, pain associated with trauma, and the chronic pain of arthritis.

Neuropathic pain, in contrast to nociceptive pain, is described as “burning”, “electric”, “tingling”, and “shooting” in nature. It can be continuous or paroxysmal in presentation. Whereas nociceptive pain is caused by the stimulation of peripheral A-delta and C-polymodal pain receptors, by inflammatory mediators, (e.g. histamine bradykinin, substance P, etc.) neuropathic pain is produced by injury or damage to peripheral nerves or the central nervous system

The hallmarks of neuropathic pain are chronic allodynia and hyperalgesia. Allodynia is defined as pain resulting from a stimulus that ordinarily does not elicit a painful response (e.g. light touch). Hyperalgesia is defined as an increased sensitivity to normally painful stimuli.

Examples of neuropathic pain include carpal tunnel syndrome, trigeminal neuralgia, post herpetic neuralgia, phantom limb pain, complex regional pain syndromes and the various peripheral neuropathies. Subsequent to nerve injury, there is increase in nerve traffic. Expression of sodium channels is altered significantly in response to injury thus leading to abnormal excitability in the sensory neurons. Nerve impulses arriving in the spinal cord stimulate the release of inflammatory protein Substance P. The presence of Substance P and other inflammatory proteins such as calcitonin gene-related peptide (CGRP) neurokinin A, vasoactive intestinal peptide removes magnesium induced inhibition and enables excitatory Inflammatory proteins such as glutamate and aspartate to activate specialized spinal cord NMDA receptors. This results in magnification of all nerve traffic and pain stimuli that arrive in the spinal cord from the periphery. in one study, monocytes/macrophages (ED-1), natural killer cells, T lymphocytes, and the pro-inflammatory cytokines tumor necrosis factor-alpha (TNF-alpha) and interleukin-6 (IL-6), were significantly produced in nerve-injured rats. Interestingly, ED-1-, TNF-alpha- and InterLeukin-6-positive cells increased more markedly in allodynic rats than in non-allodynic ones. The magnitude of the inflammatory response was not related to the extent of damage to the nerve fibers because rats with complete transection of the nerves displayed much lower production of inflammatory cytokines than rats with partial transection of the nerve⁷⁸. This is a finding commonly observed in patients where a minor injury results in severe pain that is out of proportion to the injury. Getting back to the study, the authors determined that the considerable increase in monocytes/macrophages induced by a nerve injury results in a very high release of Interleukin-6 and TNF-alpha. This may relate to the generation of touch allodynia/hyperalgesia, since there was a clear correlation between the number of ED-1 and Interleukin-6-positive cells and the degree of allodynia. Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal ‘baskets’ may underlie painful sympathetic-sensory coupling⁷⁹. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5). In another study, animals exhibiting heat hyperalgesia as a sign of neuropathic pain seven days after loose ligation of the sciatic nerve exhibited a significant increase in the concentration of brain derived neurotrophic factor (BDNF) in their lumbar spinal dorsal horn.⁸⁰ Administration of nerve growth factor to rodents has resulted in the rapid onset of hyperalgesia. In clinical trials with nerve growth factor for the treatment of Alzheimer disease and peripheral neuropathy, induction of pain has been the major adverse event⁸¹. In one study, the use of trkA-IgG, an inhibitor of Nerve Growth Factor (NGF) reduced neuroma formation and neuropathic pain in rats with peripheral nerve injury⁸² In another study, the systemic administration of anti-nerve growth factor (NGF) antibodies significantly reduced the severity of autotomy (self mutilating behavior induced by nerve damage) and prevented the spread of collateral sprouting from the saphenous nerve into the sciatic innervation territory⁸³. Activity in sympathetic fibers is associated with excessive sweating, temperature instability of the extremities and can induce further activity in sensitized pain receptors and, therefore, enhance pain and allodynia (sympathetically maintained pain). This pathologic interaction acts via noradrenaline released from sympathetic terminals and newly expressed receptors on the afferent neuron membrane⁸⁴.

Activation of motor nerves that travel from the spinal cord to the muscles results in excessive muscle tension. More inflammatory mediators are released which then excite additional pain receptors in muscles, tendons and joints generating more nerve traffic and increased muscle spasm. Persistent abnormal spinal reflex transmission due to local injury or even inappropriate postural habits may then result in a vicious circle between muscle hypertension and pain⁸⁵. Separately, constant C-fiber nerve stimulation to transmission pathways in the spinal cord results in even more release of inflammatory mediators but this time within the spinal cord. The transcription factor, nuclear factor-kappa B (NF-kappaB), plays a pivotal role in regulating the production of inflammatory cytokines⁸⁶. Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E₂ (PGE₂) levels in the cerebrospinal fluid. The major inducer of central Cox-2 upregulation is inflammatory mediator interleukin-1^{β} Din the CNS⁸⁷. Basal levels of the enzyme phospholipase A₂ activity in the CNS do not change with peripheral inflammation. The central nervous system response to pain can keep increasing even though the painful stimulus from the injured tissue remains steady. This “wind-up” phenomenon in deep dorsal neurons can dramatically increase the injured person's sensitivity to the pain.

The neurotrophins are a family of growth promoting proteins that are essential for the generation and survival of nerve cells during development, Neurotrophins promote growth of small sensory neurons and stimulate the regeneration of damaged nerve fibers They consist of four members, nerve growth factor (NGF), brain derived neurotrophic factor (BDNF), neurotrophin-3 (NT-3) and neurotrophin 4/5 (NT-4/5).

Nerve growth factor and brain-derived neurotrophic factor modulate the activity of a sodium channel (NaN) that is preferentially expressed in pain signaling neurons that innervate the body (spinal cord dorsal root ganglion neurons) and face (trigeminal neurons). Transection of a nerve fiber (axotomy) results in an increased production of inflammatory cytokines and induces marked changes in the expression of sodium channels within the sensory neurons⁸⁸. Following axotomy the density of slow (tetrodotoxin-resistant) sodium currents decrease and a rapidly repriming sodium current appears. The altered expression of sodium channels leads to abnormal excitability in the sensory neurons⁸⁹. Studies have shown that these changes in sodium channel expression following axotomy may be attributed at least in part to the loss of retrogradely transported nerve growth factor⁹⁰.

In addition to effects on sodium channels, there is a large reduction in potassium current subtypes following nerve transection and neuroma formation. Studies have shown that direct application of nerve growth factor to the injured nerve can prevent these changes⁹¹.

### Reflex Sympathetic Dystrophy/Chronic Regional Pain Syndrome (Rsd/Crps)

Reflex sympathetic dystrophy (RSD) syndrome also called Chronic Regional Pain Syndrome (CRPS) has been recognized clinically for many years. It is most often initiated by trauma to a nerve, neural plexus, or soft tissue. Diagnostic criteria are the presence of regional pain and other sensory changes following a painful injury. The pain is associated with changes in skin color, skin temperature, abnormal sweating, tissue swelling. With time, tissue atrophy may occur as well as involuntary movements, muscle spasms, or pseudoparalysis⁹². Like other organs with a blood supply, the bones also react to the disturbances in permeability caused by various inflammatory mediators. There is fluid accumulation in the bones and loss of bone density (osteoporosis)⁹³. In addition, the inflammatory mediators accelerate the rate at which bone is broken down. The bone loss is further aggravated by decreased use of the affected body part due to pain. Complex regional pain syndrome, type I (reflex sympathetic dystrophy; CRPS-I/RSD) can spread from the initial site of presentation. In one study of 27 CRPS-I/RSD patients who experienced a significant spread of pain, three patterns of spread were identified. ‘Contiguous spread (CS)’ was noted in all 27 cases and was characterized by a gradual and significant enlargement of the area affected initially. ‘Independent spread (IS)’ was noted in 19 patients (70%) and was characterized by the appearance of CRPS-I in a location that was distant and non-contiguous with the initial site (e.g. CRPS-I/RSD appearing first in a foot, then in a hand). ‘Mirror-image spread (MS)’ was noted in four patients (15%) and was characterized by the appearance of symptoms on the opposite side in an area that closely matched in size and location the site of initial presentation. Only five patients (19%) suffered from CS alone; 70% also had IS, 11% also had MS, and one patient had all three kinds of spread⁹⁴. In 1942 Paul Sudeck suggested that the signs and symptoms of RSD/CRPS including sympathetic hyperactivity might be provoked by an exaggerated inflammatory response to injury or operation of an extremity. His theory found no followers, as most doctors incorrectly believe that RSD/CRPS is solely initiated by a hyperactive sympathetic system. Recent research and studies including various clinical and experimental investigations now provide support to the theory of Paul Sudeck⁹⁵.

As we now understand, soft tissue or nerve injury causes excitation of sensory nerve fibers. Reverse (antidromic) firing of these sensory nerves causes release of the inflammatory neuropeptides at the peripheral endings of these fibers. These neuropeptides may induce vasodilation, increase vascular permeability, attract other immune cells such as T helper cells and excite surrounding sensory nerve fibers—a phenomenon referred to as neurogenic inflammation. At the level of the central nervous system, the increased input from peripheral pain receptors alters the central processing mechanisms.

Sympathetic dysfunction, which often has been purported to play a pivotal role in RSD/CRPS, has been suggested to consist of an increased rate of outgoing (efferent) sympathetic nerve impulses towards the involved extremity induced by increased firing of the sensory nerves. However, the results of several experimental studies suggest that sympathetic dysfunction also consists of super sensitivity to catecholamines induced by nerve injury (autonomic denervation)⁹⁶. Part of this occurs due to injured sensory nerves and immune cells developing receptors for the chemical transmitter norepinephrine and epinephrine (catecholamines), which are normally released by sympathetic nerves and also circulate in the blood. Stimulation of these receptors by locally released or circulating catecholamines produces sympathetic effects such as sweating, excessive hair growth and narrowing of blood vessels⁹⁷. In addition and under certain conditions, catecholamines may boost regional immune responses, through increased release of Interleukin-1, tumor necrosis factor-alpha, and Interleukin-8 production.

In several studies, patients with RSD/CRPS showed a markedly increased level of the inflammatory peptide bradykinin as well as calcitonin gene-related peptide⁹⁸. The levels of bradykinin were four times as high as the controls. A few showed increased levels of the other inflammatory chemical mediators⁹⁹. Two pain producing pathways have been identified: inflammatory stimuli induce the production of bradykinin, which stimulates the release of TNF-alpha. The TNF-alpha induces production of (i) Interleukin-6 and Interleukin-1b, which stimulate the production of cyclooxygenase products, and (ii) InterLeuken-8, which stimulates production of sympathomimetics (sympathetic hyperalgesia)¹⁰⁰.

Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. This can be induced by neurotrophins such as nerve growth factor (NGF), brain derived neurotrophic factor (BDNF), neurotrophin-3 (NT-3) and neurotrophin 4/5 (NT-4/5).

### Sports Injuries/Bursitis/Tendonitis/Rotator Cuff Tears

Inflammation of the bursa is known as bursitis. A bursa is a small sac containing fluid that lies between bone and other moving structures such as muscles, skin or tendons. The bursa allows smooth gliding between these structures. A bursa allows a tendon or muscle to move smoothly over a bone by acting as an anti-friction device and shielding the structures from rubbing against bones. Bursae are found in the knee, elbow, shoulder and wrist. If the tendons become thickened and bumpy from excessive use, the bursa is subjected to increased friction and may become inflamed. Tendonitis is inflammation or irritation of a tendon. Tendons are the thick fibrous cords that attach muscles to bone. They function to transmit the power generated by a muscle contraction to move a bone. Since both tendons and bursae are located near joints, inflammation in these soft tissues will often be perceived by patients as joint pain and mistaken for arthritis. Symptoms of bursitis and tendonitis are similar: pain and stiffness aggravated by movement. Pain may be prominent at night. Almost any tendon or bursa in the body can be affected, but those located around a joint are affected most often. The most common cause of tendonitis and bursitis is injury or overuse during work or play, particularly if the patient is poorly conditioned, has bad posture, or uses the affected limb in an awkward position. Occasionally an infection within the bursa or tendon sheath will be responsible for the inflammation. Tendonitis or bursitis may be associated with diseases such as rheumatoid arthritis, gout, psoriatic arthritis, thyroid disease and diabetes. In one study of thirty-nine patients with rotator cuff diseases, the levels of the cytokine Interleukin-1 beta was significantly correlated with the degree of pain. The combined results of immunohistochemistry indicated that both synovial lining and sublining cells produce IL-1beta, while synovial lining cells predominantly produce the anti-inflammatory intracellular InterLeukin-1 receptor antagonist (icIL-1ra) and sublining cells secrete secreted InterLeukin-1 receptor antagonist (sIL-1ra)¹⁰¹. In another study, the levels of interleukin-1 beta were significantly higher in the shoulder joints in patients with anterior instability and chronic inflammation of the joint¹⁰². In another study, immunohistological staining demonstrated the expression of Interleukin-1 beta (Interleukin-1 beta), Tumor necrosis factor alpha (TNF-alpha), transforming growth factor beta (TGF-beta), and basic fibroblast growth factor (bFGF) in subacromial bursa derived from the patients suffering from rotator cuff tear¹⁰³.

### Vulvar Vestibulitis Syndrome (VVS)/Vulvodynia

Vulvar vestibulitis syndrome is a major subtype of vulvodynia. It is a constellation of symptoms and findings involving and limited to the vulvar vestibule that consists of: (1) severe pain on vestibular touch to attempted vaginal entry, (2) tenderness to pressure localized within the vulvar vestibule, and (3) physical findings confined to vulvar erythema of various degrees. The syndrome has been seen in association with subclinical human papillomavirus, chronic recurrent candidiasis, chronic recurrent bacterial vaginosis, chronic alteration of vaginal pH, and the use of chemical and destructive therapeutic agents¹⁰⁴. In a study of VVS cases and asymptomatic controls, median tissue levels of inflammatory cytokines: IL-1 b and TNF-a, from selected regions of the vulva,, vestibule, and vagina were 2.3-fold and 1.8-fold elevated, respectively, in women with VVS compared to pain-free women. Analysis revealed a significant 2.2-fold higher median level of TNF alpha at the vulvar site compared to the vestibule. Cytokine elevations correlated poorly with inflammatory cell infiltrate and suggested cytokine production from another cell source. The study authors concluded that inflammatory cytokine elevation may contribute to the pathophysiology of mucocutaneous hyperalgesia¹⁰⁵

I claim:

1. A method for treating persistent pain disorders by inhibiting the biochemical mediators of inflammation in a subject comprising administering to said subject a therapeutically effective dosage of said inhibitor.

2. The method of claim 1, wherein the said biochemical mediator of inflammation is TNF-alpha.

3. The method of claim 1, wherein the said inhibitor is a TNF-alpha inhibitor.

4. The method of claim 1, wherein said persistent pain disorder is osteoarthritis.

5. The method of claim 1, wherein said persistent pain disorder is ligament or meniscus tear.

6. The method of claim 1, wherein said persistent pain disorder is neurogenic inflammation.

7. The method of claim 1, wherein said persistent pain disorder is muscle inflammation

8. The method of claim 1, wherein said persistent pain disorder is back or neck pain arising from injury to the nerve, muscle, joint, ligament or disk.

9. The method of claim 1, wherein said persistent pain disorder is neck pain arising from injury to the muscle, joint, ligament or disk.

10. The method of claim 1, wherein said persistent pain disorder is interstitial cystitis.

11. The method of claim 1, wherein said persistent pain disorder is migraine.

12. The method of claim 1, wherein said persistent pain disorder is neuropathic pain syndrome including neuralgia or nerve pain, carpal tunnel syndrome, post herpetic neuralgia, phantom limb pain, vulvodynia.

13. The method of claim 1, wherein said persistent pain disorder is chronic regional pain syndrome also known as reflex sympathetic dystrophy.

14. The method of claim 1, wherein said persistent pain disorder is bursitis including rotator cuff bursitis.

15. The method of claim 1, wherein said persistent pain disorder is tendonitis.

16. The method of claim 1, wherein said TNF-.alpha. inhibitor is administered systemically or locally.

17. The method of claim 1, wherein said TNF-.alpha inhibitor is administered parenterally.

18. The method of claim 1, wherein said TNF-.alpha inhibitor is administered intramuscularly, intravenously, by intra-articular injection, subcutaneously, orally, or rectally.

19. The method of claim 15, wherein said TNF-.alpha inhibitor is administered intravenously by injection or infusion wherein said dosage level is in the range of 2.5 mg/kg to 20 mg/kg.

20. The method of claim 15, wherein said TNF-.alpha inhibitor is administered intramuscularly wherein said dosage level is in the range of 25 mg to 100 mg.

21. The method of claim 15, wherein said TNF-alpha inhibitor is administered orally at a dosage of about 20 mg to about 1,500 mg.

22. The method of claim 15, wherein said TNF-.alpha inhibitor is administered subcutaneously wherein said dosage level is in the range of 5 mg to 50 mg for acute or chronic regimens

23. The method of claim 15, wherein said TNF-.alpha inhibitor is administered by intra-articular injection wherein said dosage level is in the range of 25 mg to 100 mg.

24. The method of claim 15, wherein said TNF-.alpha inhibitor is administered intranasally wherein said dosage level is in the range of 0.1 mg to 10 mg for acute or chronic regimens

25. The method of claim 1, wherein the TNF-.alpha inhibitor is selected from the group consisting of etanercept, infliximab, CDP571 (a humanized monoclonal anti-TNF-alpha antibody), pegylated soluble TNF receptor Type I (PEGsTNF-R1), D2E7, Thalidomide based compounds, Pentoxifylline and Phosphodiesterase inhibitors.

26. The method of claim 1, wherein the said biochemical mediator of inflammation is Interleukin-1.

27. The method of claim 1, wherein the said inhibitor is an Interleukin-1 receptor antagonist.

28. The method of claim 1, wherein said persistent pain disorder is osteoarthritis.

29. The method of claim 1, wherein said persistent pain disorder is ligament or meniscus tear.

30. The method of claim 1, wherein said persistent pain disorder is neurogenic inflammation.

31. The method of claim 1, wherein said persistent pain disorder is muscle inflammation.

32. The method of claim 1, wherein said persistent pain disorder is back pain arising from injury to the nerve, muscle, joint, ligament or disk.

33. The method of claim 1, wherein said persistent pain disorder is neck pain arising from injury to the muscle, joint, ligament or disk.

34. The method of claim 1, wherein said persistent pain disorder is interstitial cystitis.

35. The method of claim 1, wherein said persistent pain disorder is migraine.

36. The method of claim 1, wherein said persistent pain disorder is neuropathic pain syndrome including neuralgia or nerve pain, carpal tunnel syndrome, post herpetic neuralgia, phantom limb pain, vulvodynia.

37. The method of claim 1, wherein said persistent pain disorder is chronic regional pain syndrome also known as reflex sympathetic dystrophy.

38. The method of claim 1, wherein said persistent pain disorder is bursitis including rotator cuff bursitis.

39. The method of claim 1, wherein said persistent pain disorder is tendonitis.

40. The method of claim 1, wherein said Interleukin-1 receptor antagonist is administered systemically or locally.

41. The method of claim 1, wherein said Interleukin-1 receptor antagonist is administered parenterally.

42. The method of claim 1, wherein said Interleukin-1 receptor antagonist is administered intramuscularly, intravenously, by intra-articular injection, subcutaneously, orally, or rectally.

43. The method of claim 15, wherein said Interleukin-1 receptor antagonist is administered intravenously by injection or infusion wherein said dosage level is in the range of 2.5 mg/kg to 20 mg/kg.

44. The method of claim 15, wherein said Interleukin-1 receptor antagonist is administered intramuscularly wherein said dosage level is in the range of 25 mg to 100 mg.

45. The method of claim 15, wherein said Interleukin-1 receptor antagonist is administered orally at a dosage of about 20 mg to about 1,500 mg.

46. The method of claim 15, wherein said Interleukin-1 receptor antagonist is administered subcutaneously wherein said dosage level is in the range of 5 mg to 50 mg for acute or chronic regimens

47. The method of claim 15, wherein said Interleukin-1 receptor antagonist is administered by intra-articular injection wherein said dosage level is in the range of 25 mg to 100 mg.

48. The method of claim 15, wherein said Interleukin-1 receptor antagonist is administered intranasally wherein said dosage level is in the range of 0.1 mg to 10 mg for acute or chronic regimens

49. The method of claim 1, wherein the Interleukin-1 receptor antagonist is selected from the group consisting of naturally occurring and Human recombinant Interleukin-1 receptor antagonist.

50. The method of claim 1, wherein the said biochemical mediator of inflammation is leukotriene

51. The method of claim 1, wherein the said inhibitor is a leukotriene receptor antagonist.

52. The method of claim 1, wherein said leukotriene receptor antagonist is administered intramuscularly, intravenously, by intra-articular injection, subcutaneously, orally, or rectally.

53. The method of claim 1, wherein the said biochemical mediator of inflammation is 5-lipoxygenase.

54. The method of claim 1, wherein the said inhibitor is a 5-lipoxygenase antagonist

55. The method of claim 1, wherein said 5-lipoxygenase antagonist is administered intramuscularly, intravenously, by intra-articular injection, subcutaneously, orally, or rectally.

56. The method of claim 1, wherein the said biochemical mediator of inflammation is nitric oxide

57. The method of claim 1, wherein the said inhibitor is a nitric oxide antagonist and is selected from the group including Oxcarbazepine, Carbamazepine and Zonisamide.

58. The method of claim 1, wherein said nitric oxide antagonist is administered intramuscularly, intravenously, by intra-articular injection, subcutaneously, orally, or rectally.

59. The method of claim 1, wherein the said biochemical mediator of inflammation is Substance P.

60. The method of claim 1, wherein the said inhibitor is a Substance P antagonist and is selected from the group including corticosteroids, Ondansetron and 5-HT3-receptor antagonists.

61. The method of claim 1, wherein said Substance P antagonist is administered intramuscularly, intravenously, by intra-articular injection, subcutaneously, orally, or rectally.

62. The method of claim 1, wherein the said biochemical mediator of inflammation is calcitonin gene-related peptide.

63. The method of claim 1, wherein the said inhibitor is a calcitonin gene-related peptide antagonist.

64. The method of claim 1, wherein said calcitonin gene-related peptide antagonist is administered intramuscularly, intravenously, by intra-articular injection, subcutaneously, orally, or rectally.

65. The method of claim 1, wherein the said biochemical mediator of inflammation is vasoactive intestinal peptide.

66. The method of claim 1, wherein the said inhibitor is a vasoactive intestinal peptide antagonist and is selected from the group including Botulinum toxin.

67. The method of claim 1, wherein said vasoactive intestinal peptide antagonist is administered intramuscularly, intravenously, by intra-articular injection, subcutaneously, orally, or rectally.

68. The method of claim 1, wherein the said biochemical mediator of inflammation is interleukin-4.

69. The method of claim 1, wherein the said inhibitor is an interleukin-4 antagonist.

69. The method of claim 1, wherein said interleukin-4 antagonist is administered intramuscularly, intravenously, by intra-articular injection, subcutaneously, orally, or rectally.

70. The method of claim 1, wherein the said biochemical mediator of inflammation is interleukin-6.

71. The method of claim 1, wherein the said inhibitor is an interleukin-6 antagonist and is selected from the group including bisphosphonates.

72. The method of claim 1, wherein said interleukin-6 antagonist is administered intramuscularly, intravenously, by intra-articular injection, subcutaneously, orally, or rectally.

73. The method of claim 1, wherein the said biochemical mediator of inflammation is interleukin-8.

74. The method of claim 1, wherein the said inhibitor is an interleukin-8 antagonist.

75. The method of claim 1, wherein said interleukin-8 antagonist is administered intramuscularly, intravenously, by intra-articular injection, subcutaneously, orally, or rectally.

76. The method of claim 1, wherein the said biochemical mediator of inflammation is a kinin.

77. The method of claim 1, wherein the said inhibitor is a kinin antagonist.

78. The method of claim 1, wherein said kinin antagonist is administered intramuscularly, intravenously, by intra-articular injection, subcutaneously, orally, or rectally.

79. The method of claim 1, wherein the said biochemical mediator of inflammation is serotonin.

80. The method of claim 1, wherein the said inhibitor is a serotonin receptor antagonist.

81. The method of claim 1, wherein said serotonin receptor antagonist is administered intramuscularly, intravenously, by intra-articular injection, subcutaneously, orally, or rectally.

82. The method of claim 1, wherein the said biochemical mediator of inflammation is Matrix Metallo-Proteinase.

83. The method of claim 1, wherein the said inhibitor is a Matrix Metallo-Proteinase antagonist and is selected from the group including Tetracyclines and macrolide antibiotics such as Clarithromycin.

84. The method of claim 1, wherein said Matrix Metallo-Proteinase antagonist is administered intramuscularly, intravenously, by intra-articular injection, subcutaneously, orally, or rectally.
